(19) United States
(12) Patent Application Publication
(10) Pub. No.: US 2012/0109266 A1
(43) Pub. Date: May 3, 2012

(54) DEVICE FOR HEATING SKIN

(76) Inventor: Amir Waldman, Yarqona (IL)

(21) Appl. No.: 12/912,987

(22) Filed: Oct. 27, 2010

(51) Int. Cl. A61N 5/06; A61F 7/00

(52) U.S. Cl. 607/90

(57) ABSTRACT

A device for heating skin including an energy source for generating heat and light energy, a reflector arranged to direct heat generated by the energy source towards a skin interface element, the skin interface element having a skin interface area sufficient for producing a heat penetration depth into skin in accordance with a treatment plan, and wherein the skin interface element includes one or more light conduits for light energy generated by the energy source to pass therethrough to skin and one or more elements for conducting heat energy generated by the energy source to the skin, wherein the light conduits are formed as at least two separate patterns separated by at least one gap surface, the gap surface arranged to direct thermal energy to the skin.

### FIELD OF THE INVENTION

The present invention relates to devices and method for heating skin, such as for treatment of skin and for skin hair permanent removal, skin rejuvenation.

### BACKGROUND OF THE INVENTION

Treatment of the upper layer of the skin, epidermis and dermis is performed in order to achieve younger and nice appearance of the skin. Light therapy is a very effective tool in addressing a variety of lesions in the skin such as pigmented and vascular lesions, and hair follicles. Both laser light and incoherent light energy have been suggested for use, wherein pulsed optical energy heats the selected lesion without damaging the surrounding tissue. In order to selectively heat the lesion, the optical spectrum, pulse duration, energy density, and spot size have to be carefully set. Existing systems use pulse durations in the range of 2-100 msec and energy density of 10-60 J/cm².

Removal of excess or otherwise undesired hair from different parts of the human body has become more and more popular, particularly for cosmetic and aesthetic reasons.

### SUMMARY OF THE INVENTION

The present invention seeks to provide devices and method for heating skin, as is described more in detail hereinbelow. The invention may be used in a variety of dermatological applications, such as but not limited to, epilation, treatment of superficial vascular lesions, benign pigmented lesions, acne or bromidrosis, and for removal of unwanted hair, for example. The apparatus of the present invention may be domestically used by all patients with no prior professional/training.

There is thus provided in accordance with an embodiment of the present invention a device for heating skin including an energy source for generating heat and light energy, a reflector arranged to direct heat generated by the energy source towards a skin interface element, the skin interface element having a skin interface area sufficient for producing a heat penetration depth into skin in accordance with a treatment plan, and wherein the skin interface element includes one or more light conduits for light energy generated by the energy source to pass therethrough to skin and one or more elements for conducting heat energy generated by the energy source to the skin, wherein the light conduits are formed as at least two separate patterns separated by at least one gap surface, the gap surface arranged to direct thermal energy to the skin.

### BRIEF DESCRIPTION OF THE DRAWINGS

These and additional constructional features and advantages of the invention will be more readily understood in the light of the ensuing description of embodiments thereof, given by way of example only, with reference to the accompanying drawings wherein:

FIGS. 1A and 1B are simplified partially cutaway and pictorial illustrations, respectively, of a device for heating skin, constructed and operative in accordance with an embodiment of the present invention.

### DETAILED DESCRIPTION OF EMBODIMENTS

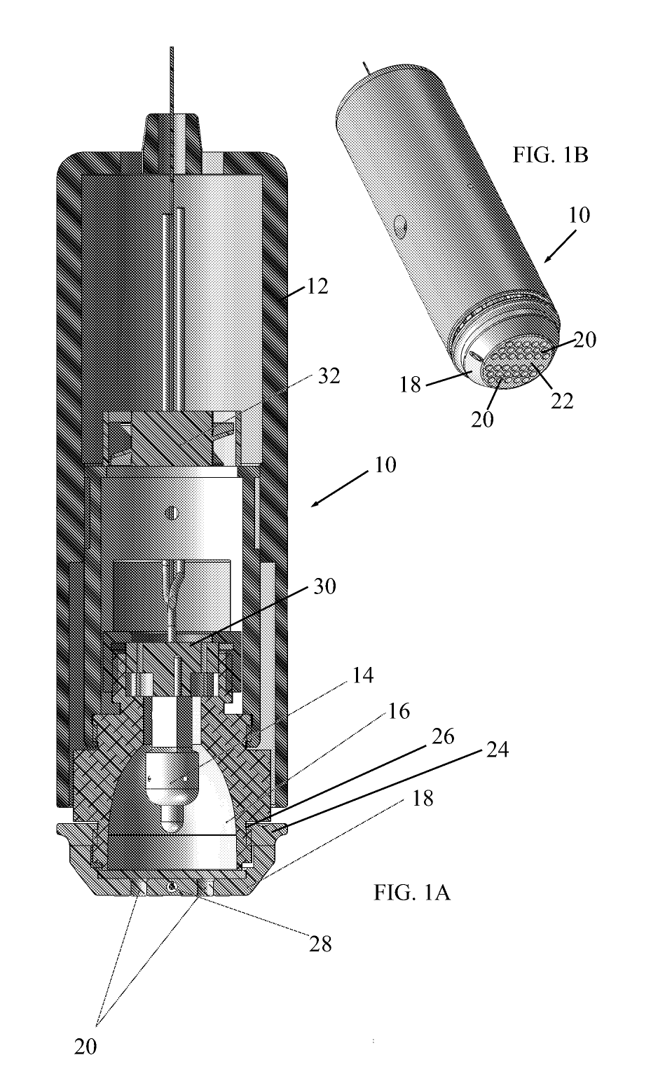

Reference is now made to FIGS. 1A and 1B, which illustrate a device 10 for heating skin, constructed and operative in accordance with a non-limiting embodiment of the present invention.

Device 10 includes a housing 12, which may be cylindrical in shape and molded of an engineering plastic, although other shapes and materials are also within the scope of the invention. Device 10 includes a heat source 14 (also referred to as energy source 14), which in the illustrated non-limiting embodiment, is a halogen lamp (e.g., a 12V, 20 W halogen lamp). Other sources of heat may be used instead of a halogen lamp, such as but not limited to, electrical, optical and chemical sources of heat and any combination thereof. In the case of optical energy, the thermal energy may be produced by an incandescent lamp, LED, laser, or gas filled lamp in the visible and infrared ranges, for example. (The illustrated embodiment shows an electrical wire for providing power to heat source 14 and other components of the device 10, but internal power sources such as batteries are also contemplated.)

The heat generated by heat source 14 is concentrated and directed by a reflector 16, such as but not limited to, a parabolic reflector symmetrically arranged with heat source 14 such that heat source 14 is situated along the longitudinal axis of reflector 16. Reflector 16, which may be a polished metal surface, for example, directs the thermal energy towards a skin interface element 18. The choice of whether reflector 16 is reflective (that is, substantially non-diffusive) or diffusive may be based on the particular application and treatment needs.

As mentioned before, heat source 14 may also be a light source (as in the illustrated embodiment). In such an embodiment, reflector 16 also directs the light towards skin interface element 18. The light can pass through a plurality of light conduits 20 formed through skin interface element 18. Light conduits 20 may be simply apertures drilled or otherwise formed through skin interface element 18, or glass tubes or transparent windows that serve as light guides. In the illustrated embodiment, light conduits 20 are formed as at least two separate patterns separated by one or more gap surfaces 22. The gap surface 22 contacts the skin and serves to direct thermal energy to the skin. In this manner, a combination of light and thermal energy is directed to the skin. Thus, skin interface element 18 includes one or more transparent elements (light conduits 20) for light energy to pass therethrough and one or more elements (the other surfaces of skin interface element 18, such as gap surface 22, which may or may not be opaque) for conducting heat energy to the skin (and which light energy cannot pass therethrough in the case of being opaque).

The light emitted by light source 14 may be continuous or pulsed. For example, in accordance with a non-limiting embodiment of the invention, the wavelength of the light emitted by light source 14 may be in the range of about 600-10,000 nm either CW (continuous wave) or with a pulse width of about 10 msec-1 sec, and an energy fluence of about 20-10,000 J/cm². The light may be delivered as a single pulse at one place on the skin or a series of pulses at one place on the skin before moving to other treatment sites on the skin.

Skin interface element 18 has a skin interface area sufficient for producing a heat penetration depth into the skin in accordance with a treatment plan. Skin interface element 18 is made from a material that has a thermal conductivity higher than the thermal conductivity of the skin (virtually any metal satisfies this criterion, and even some plastics). In one embodiment, skin interface element 18 is made of a metal with good thermal conductivity, such as an aluminum alloy. In such an embodiment, there is no detectable thermal gradient across the face of skin interface element 18. However, other embodiments are also contemplated wherein skin interface element 18 is made of metals such as stainless steel alloys or non-metals with thermal conductivity one-tenth and even less than aluminum alloys. Such an embodiment provides unique treatment possibilities, wherein the thermal energy provided to the skin at gap surface 22 is different than the thermal energy provided to the skin at other portions of skin interface element 18.

Skin interface element 18 may be applied directly to the skin or alternatively through conducting media, such as gel, cream and the like.

Skin interface element 18 may be fixedly attached to the body of reflector 16. In the illustrated embodiment, skin interface element 18 has a peripheral lip 24 which slidingly fits into a circumferential groove 26 formed on the outside of the body of reflector 16. In this manner, skin interface element 18 can be rotated with respect to reflector 16. In this manner, the light conduits 20 and gap surface 22 can be oriented at different orientations with respect to reflector 16 and housing 12 (which may have significance, for example, if reflector 16 is not symmetrical with respect to heat source 14).

A temperature sensor 28 may be assembled on any portion of skin interface element 18 (e.g., embedded therein or attached thereon) for detecting the temperature generated thereat. Temperature sensor 28 may be, without limitation, a thermistor (positive or negative coefficient), thermo-transistor, thermocouple, IR temperature element, and others. Temperature sensor 28 may operate in a control loop with control circuitry 30 to control or shut down operation of heat source 14 in accordance with the feedback temperature sensed.

An internal fan 32 may be provided for cooling heat source 14 and other components of the device 10. Fan 32 can direct cooling air to cool skin interface element 18 for faster thermal response when adjusting the heat power generated by heat source 14.

Device 10 may be used for collagen remodeling, such as but not limited to, skin rejuvenation, skin tightening, removal of wrinkles, tightening or cleaning pores, improving skin texture and others.

In one non-limiting embodiment of the invention, skin treatment (e.g., collagen remodeling) includes the following steps:

a. Applying heat from heat source 14 to skin interface element 18 which transfers heat to the skin surface.

b. Measuring temperature of skin interface element 18 with temperature sensor 28.

c. Controlling heat power from heat source 14 according to feedback obtained from temperature sensor 28

d. Cooling skin interface element 18 with fan 32 for quick thermal response while changing the heat power generated by heat source 14.

In another non-limiting embodiment of the invention, the skin treatment (e.g., collagen remodeling) includes irradiating the skin using optical energy delivered from energy source 14 via skin interface element 18, wherein optical energy is delivered through one or more transparent surfaces of skin interface element 18 (e.g., light conduits 20) and thermal energy is delivered via one or more surfaces of skin interface element 18 (e.g., gap surface 22) through which heat is conducted to the skin.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A device for heating skin comprising:
an energy source for generating heat and light energy;
a reflector arranged to direct heat generated by said energy source towards a skin interface element, said skin interface element having a skin interface area sufficient for producing a heat penetration depth into skin in accordance with a treatment plan; and
wherein said skin interface element comprises one or more light conduits for light energy generated by said energy source to pass therethrough to skin and one or more elements for conducting heat energy generated by said energy source to the skin, wherein said light conduits are formed as at least two separate patterns separated by at least one gap surface, said gap surface arranged to direct thermal energy to the skin.

2. The device according to claim 1, wherein said light conduits are apertures formed through said skin interface element.

3. The device according to claim 1, wherein said light conduits comprise transparent windows.

4. The device according to claim 1, wherein said one or more elements for conducting heat energy are opaque.

5. The device according to claim 1, further comprising a temperature sensor assembled on a portion of said skin interface element for detecting temperature generated thereat.

6. The device according to claim 5, wherein said temperature sensor operates in a control loop with control circuitry to control operation of said energy source in accordance with feedback temperature sensed by said temperature sensor.

7. The device according to claim 1, wherein said skin interface element is made from a material that has a thermal conductivity higher than the thermal conductivity of the skin.

8. The device according to claim 1, wherein said skin interface element is made from a material that has a thermal conductivity such that thermal energy provided to the skin at said gap surface is different than thermal energy provided to the skin at other portions of said skin interface element.

9. The device according to claim 1, wherein said skin interface element is rotatable with respect to said reflector.

10. A method for heating skin comprising:
applying heat from an energy source to a skin interface element which transfers heat to a skin surface;
measuring temperature of said skin interface element with a temperature sensor;
controlling heat from said energy source according to feedback obtained from said temperature sensor; and
cooling said skin interface element with a fan for quick thermal response while changing heat generated by said energy source.

11. A method for heating skin comprising:
irradiating skin using optical energy delivered from an energy source via a skin interface element, wherein the optical energy is delivered through one or more transparent surfaces of said skin interface element and thermal energy is delivered via one or more surfaces of said skin interface element through which heat is conducted to the skin.
